(19) United States
(12) Patent Application Publication
(10) Pub. No.: US 2025/0242062 A1
(43) Pub. Date: Jul. 31, 2025

(54) ANTI-COX-2 NANOBODIES FOR ENDOSCOPIC VISUALIZATION OF COLORECTAL ADENOMAS

(71) Applicant: Vanderbilt University, Nashville, TN (US)

(72) Inventors: Md. Jashim UDDIN, Nashville, TN (US); Shu XU, Nashville, TN (US); Ansari M. Aleem, Nashville, TN (US); Brian E. WADZINSKI, Nashville, TN (US); Benjamin W. SPILLER, Nashville, TN (US); Lawrence J. MARNETT, Nashville, TN (US); Michael Christopher GOODMAN, Nashville, TN (US)

(21) Appl. No.: 18/855,486

(22) PCT Filed: Mar. 30, 2023
(86) PCT No.: PCT/US2023/065120

§ 371 Date: Oct. 9, 2024

Related U.S. Application Data
(60) Provisional application No. 63/329,587, filed on Apr. 11, 2022.

(51) Int. Cl. A61K 49/00; A61B 1/04; A61B 1/31

(52) U.S. Cl. CPC A61K 49/006; A61B 1/043; A61B 1/046; A61B 1/31

(57) ABSTRACT

Colorectal cancer (CRC) is one of the leading causes of cancer-related mortality in men and women. Uncontrolled proliferation of CRC cells is pivotal in colorectal tumorigenesis and cyclooxygenase-2 (COX-2) is an important regulatory enzyme in this process. Timely diagnosis is the key to life-saving management of CRC, which is under-diagnosed because colorectal aberrant crypt foci, hyper plastic polyps, micro edemas are often missed with conventional colonoscopy. Disclosed herein are imaging agents tagged to nanobodies specific for COX-2 protein that can selectively accumulate and retain in COX-2 expressing cells and colorectal adenomas, resulting in specific and sensitive of early detection, while reducing background and off-target systemic toxicity. The prospect of combining structural and functional imaging readouts obtained with fluorescence-aided colonoscopy measurements would provide powerful diagnostic information for improved management of colorectal carcinogenesis.

### CROSS-REFERENCE TO RELATED APPLICATIONS

This application claims benefit of U.S. Provisional Application No. 63/329,587, filed Apr. 11, 2022, which is hereby incorporated herein by reference in its entirety.

### STATEMENT OF GOVERNMENT INTEREST

This invention was made with Government Support under Grant Nos. CA128323, CA260958, and CA89450 awarded by the National Institutes of Health. The Government has certain rights in the invention.

### SEQUENCE LISTING

This application contains a sequence listing filed in ST.26 format entitled “222230_2160_Sequence_Listing” created on Mar. 6, 2023. The content of the sequence listing is incorporated herein in its entirety.

### BACKGROUND OF THE INVENTION

Precursor lesions such as aberrant crypt foci, hyperplastic polys, and macro- or micro-adenomas are signatures of abnormal proliferative activity of colorectal epithelium. Morphologically, these often small and flat precursors and associated lesions are different from the normal colonic mucosa, but under white light, they are difficult to distinguish from surrounding healthy tissue. Colonoscopic detection and removal of these pre-neoplastic lesions followed by surveillance represent the most important prevention method for colorectal cancer (CRC).

Endoscopic imaging strategies have significantly impact in preclinical studies of CRC. Current investigations of molecular mechanisms in animal models often involve endpoint measurements on excised tissues. However, animal-to-animal variations in the timing of disease progression and extent of severity often make it preferable to perform longitudinal measurements of molecular expression within the same animal over time. Endoscopic molecular imaging technologies are needed enable these types of measurements to be performed.

### SUMMARY OF THE INVENTION

Disclosed herein are imaging agents tagged to nanobodies specific for COX-2 protein that can selectively accumulate in colorectal adenomas, resulting in specific and sensitive of early detection, while reducing background and off-target systemic toxicity. The prospect of combining structural and functional imaging readouts obtained with fluorescence-aided colonoscopy measurements would provide powerful diagnostic information for improved management of colorectal carcinogenesis. For example, with these approaches, initiation of CRC or in other word colorectal adenomas revealed by COX-2 expression could be coupled with imaging approaches targeted fluorescent COX-2 nanobodies, for early diagnosis and intervention with appropriate cell-or molecularly-directed therapies.

The disclosed A7 nanobody exhibits high affinity for COX-2 and offers significant advantages over the existing COX-2-targeted imaging agents developed for in vivo diagnostic imaging. It exhibits high affinity and specificity for COX-2, ease of production and manipulation, excellent stability, and substantial water solubility. In addition to low immunogenicity, this COX-2-targeted nanobody exhibits excellent cell penetrance property as it is one-tenth of the size of a polyclonal COX-2 antibody. The structural and functional basis of COX-2 binding affinity of the disclosed nanobody was determined (molecular weight: 15.24 kDa). The fluorescently (NIR664, excitation 670 nm, emission 690 nm) labeled A7 nanobody (A7-NIR664) administered intravenously enabled detection of colorectal adenoma lesions in the colon without penetrating into normal colon tissues beyond the adenoma lesions. The fluorescently-labeled COX-2-specific nanobody is non-toxic to cells and tissues, highly-resistant to peptidases, stable in serum, and capable of imaging COX-2 in colorectal adenomas with high specificity, sensitivity and signal to noise ratios (SNR).

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1A is a schematic representation of the development of anti-COX-2 nanobodies. FIG. 1B shows transformation of A7 nanobody (SEQ ID NO:59) into A7-NIR664 nanobody conjugate using a protein bioconjugate chemistry.

FIG. 2 shows dissociation constant while bound to purified COX-1 and COX-2 along with the COX-2 inhibition IC50 value of purified A7 nanobody.

FIG. 3A is a schematic of AOM and DSS treatment procedure for the development of colorectal adenomas in B6;129 mice. FIG. 3B is a photograph of an excised colon containing adenomas. FIG. 3C shows tumor penetrance induced by AOM/DSS treatment. FIG. 3D shows white-light colonoscopy of adenomas at 10 weeks post-AOM/DSS treatment in B6;129 mice.

FIG. 4A contains representative white-light and fluorescence in vivo colonoscopy images of B6;129 mice bearing AOM/DSS-induced colorectal adenomas or mice with healthy colon dosed with A7-NIR664 nanobody (4 mg/kg, i.v.) at 1 h post-injection. FIG. 4B shows colonoscopy image analysis of colorectal adenomas of AOM/DSS B6; 129 mice dosed with F9-A7-NIR664 at 1 h post-injection using ImageJ software.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

As used herein, the terms “single domain antibody (VHH)” and “nanobodies” have the same meaning referring to a variable region of a heavy chain of an antibody, and construct a single domain antibody (VHH) consisting of only one heavy chain variable region. It is the smallest antigen-binding fragment with complete function. The nanobody may be produced by any means. For instance, the nanobody may be enzymatically or chemically produced by fragmentation of an intact antibody, it may be recombinantly produced from a gene encoding the partial antibody sequence, or it may be wholly or partially synthetically produced.

The term “antigen binding site” refers to a region of an antibody that specifically binds an epitope on an antigen.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

A “fusion protein” or “fusion polypeptide” refers to a hybrid polypeptide which comprises polypeptide portions from at least two different polypeptides. The portions may be from proteins of the same organism, in which case the fusion protein is said to be “intraspecies”, “intragenic”, etc. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. A first polypeptide may be fused to the N-terminus, the C-terminus, or the N- and C-terminus of a second polypeptide. Furthermore, a first polypeptide may be inserted within the sequence of a second polypeptide.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-α-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “specifically binds”, as used herein refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “cell penetrating peptide”, “cell penetrating protein”, “CPP” and the like, as used herein, refer to a peptide or protein having an ability to pass through cellular membranes. In various embodiments, a CPP is conjugated to a nanobody disclosed herein to facilitate transport of the nanobody across the membrane. In some embodiments, a CPP is capable of being internalized into a cell and passing cellular membranes (including, inter alia, the outer “limiting” cell membrane (also commonly referred to as “plasma membrane”), endosomal membranes, and membranes of the endoplasmatic reticulum). In some embodiments, any possible mechanism of internalization is envisaged including both energy-dependent (i.e. active) transport mechanisms (e.g., endocytosis) and energy-independent (i.e. passive) transport mechanism (e.g., diffusion).

### Nanobodies

Disclosed are compositions and methods for endoscopic visualization of colorectal adenomas. Specifically, disclosed herein is a COX-2-specific nanobody conjugated to a detectable moiety.

In some embodiments, the nanobody comprises a variable domain having CDR1, CDR2 and CDR3 sequences. For example, in some embodiments, the CDR1 sequence comprises the amino acid sequence GSIFSINVM (SEQ ID NO:1); CDR2 sequence of the variable domain comprises the amino acid sequence ELVATITSGGTTNY (SEQ ID NO:2); and the CDR3 sequence of the variable domain comprises the amino acid sequence VYYCNAKDLGGSSWFSEFDY (SEQ ID NO:3). In some embodiments, the nanobody has one or more conservative substitutions in SEQ ID NOs: 1, 2, and/or 3.

In some embodiments, the disclosed nanobody has the amino acid sequence: QVQLQESGGGLVQPGGSLKLSCAASGSIFSINVMGWYRQAPGKQRELVATITSGGTTNYADSV KGRFTISRDNAKDTLYLQMNSLKPEDTAVYYCNAKDLGGSSWFSEFDYWGQGTQVTVGPGGQ (SEQ ID NO:4), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:4.

In some embodiments, the disclosed nanobody comprises other moieties, such as a HIS tag. For example, the disclosed nanobody can have the amino acid sequence:

(SEQ ID NO: 5)
QVQLQESGGGLVQPGGSLKLSCAASGSIFSINVMGWYRQAPGKQRELVAT
ITSGGTTNYADSVKGRFTISRDNAKDTLYLQMNSLKPEDTAVYYCNAKDL
GGSSWFSEFDYWGQGTQVTVGPGGQHHHHHHGAYPYDVPDYAS.

Therefore, in some embodiments, the nanobody is encoded by the nucleic acid sequence

(SEQ ID NO: 6)
atggcccaggtgcagctgcaggagtctggaggaggcttggtgcagcctgg
ggggtctctgaagctctcctgtgcagcctctggaagcatcttcagtatca
atgtcatgggctggtaccgccaggctccagggaagcagcgcgagttggtc
gcaactattactagtggtggtaccacaaactatgcagactccgtgaaggg
ccgattcaccatctccagagacaacgccaaggacacgctgtatctgcaaa
tgaacagcctgaaacctgaggacacggccgtctattactgtaatgccaaa
gacttgggcggtagtagctggtttagtgagtttgactactggggccaggg
aacccaggtcaccgtcggcccgggaggccaacaccatcaccaccatcatg
gcgcatatccgtatgatgtgccggactatgcttctt.

In the present invention, “a conservative substitution” refers to 1, 2, 3, 4, 5, or 6 amino acids substituted by amino acids having analogical or similar properties, compared to the amino acid sequence of the nanobody of the present invention. These conservative substitutions can, for example, be produced according to the amino acid substitutions in

TABLE 1
Table 1. Conservative Substitutions

| Original residue | substitution | substitution |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

Also disclosed herein is a fusion protein comprising the disclosed nanobodies or fragments thereof. In addition to almost full-length polypeptides, the present invention also includes fragments of the nanobodies of the invention. Typically, the fragment has at least about 50 contiguous amino acids of the disclosed nanobody, preferably at least about 50 contiguous amino acids, more preferably at least about 80 contiguous amino acids, and most preferably at least about 100 contiguous amino acids.

In some embodiments, the nanobody disclosed herein is conjugated to a cell penetrating peptide (CPP). Non-limiting examples of CPPs include the HIV-1 TAT translocation domain (Green; M. and Loewenstein, P. M. (1988) Cell 55, 1179-1188) and the homeodomain of the Antennapedia protein from Drosophila (Joliot; A. et al. (1991) Proc. Natl. Acad. Sci. USA 88, 1864-1868); a sequence of 16 amino acids called penetratin or pAntp of the Antennapedia protein (Derossi, D. et al. (1994) J. Biol. Chem. 269, 10444-10450); a basic sequence of the HIV-1 Tat protein (Vives, E. et al. (1997) J. Biol. Chem. 272, 16010-16017); and a synthetic peptide developed is the amphipathic model peptide MAP (Oehlke, J. et al. (1998) Biochim. Biophys. Acta 1414, 127-139). Additional non-limiting examples of CPPs are described in U.S. Pat. Nos. 9,303,076; and 9,302,014. Examples of linear CPPs are provided in Table 2.

TABLE 2
Linear CPPs

| | | |
|---|---|---|
| cpp1 | IRRGISRK | SEQ ID NO: 7 |
| cpp2 | KRKRAV | SEQ ID NO: 8 |
| cpp3 | PKPKRQTKR | SEQ ID NO: 9 |
| cpp4 | RRRRHCNR | SEQ ID NO: 10 |
| cpp5 | IPDPTGQS | SEQ ID NO: 11 |
| cpp6 | IKREREND | SEQ ID NO: 12 |
| cpp7 | KKLQEQEKQQKVEFRKR | SEQ ID NO: 13 |
| cpp8 | GPNKKKRKL | SEQ ID NO: 14 |
| cpp9 | RRRRASAPISQWSSSRRSR | SEQ ID NO: 15 |
| cpp10 | KLALKLALKALKAALKLA | SEQ ID NO: 16 |
| cpp11 | FLPLIGRVLSGIL | SEQ ID NO: 17 |
| cpp12 | CVQWSLLRGYQPCCVQWSLLRGYQPC | SEQ ID NO: 18 |
| cpp13 | RKKRKGSGSRKKRKGSGSRKKRKGSGSRKKRK | SEQ ID NO: 19 |
| cpp14 | CVQWSLLRGYQPCGSGSCVQWSLLRGYQPC | SEQ ID NO: 20 |
| cpp15 | KKRRRGSGSKKRRRGSGSKKRRRGSGSKKRRR | SEQ ID NO: 21 |
| cpp16 | RKKRRGSGSRKKRRGSGSRKKRRGSGSRKKRR | SEQ ID NO: 22 |
| cpp17 | RQIKIWFQNRRMKWKKC | SEQ ID NO: 23 |
| [GRKRKRS]4 | GRKRKRSGRKRKRSGRKRKRSGRKRKRS | SEQ ID NO: 24 |
| (PRKKRGR]4 | PRKKRGRPRKKRGRPRKKRGRPRKKRGR | SEQ ID NO: 25 |
| CPP Motif 1 | CVQWSLLRGYQPC | SEQ ID NO: 26 |
| CPP Motif 2 | XKXRX-GSGS, where X is R or K | SEQ ID NO: 27 |
| 6xHis | HHHHHH | SEQ ID NO: 28 |
| HIV-TAT | RKKRRQRRR | SEQ ID NO: 29 |
| HIV-REV | RQARRNRRRRWR | SEQ ID NO: 30 |
| NLS | PKKKRKV | SEQ ID NO: 31 |
| dodecahistidine | HHHHHHHHHHHH | SEQ ID NO: 32 |

In some embodiments, the CPP is a cyclic CPP. Examples of cyclic CPPs are described in US/20210070806, which is incorporated by references in its entirety for the teaching of these CPPs. Examples of cyclic CPPs are provided in Table 3.

TABLE 3
cyclic CPPs

| | | |
|---|---|---|
| CPP1 | cyclo(FϕRRRRQ) | SEQ ID NO: 33 |
| CPP9 | cyclo(fϕRrRrQ) | SEQ ID NO: 34 |
| CPP12 | cyclo(FfϕRrRrQ) | SEQ ID NO: 35 |
| CPP1-1 | cyclo(FtBuRRRRQ) | SEQ ID NO: 36 |
| CPP1-2 | cyclo(DapHexanRRRRQ) | SEQ ID NO: 37 |
| CPP1-3 | cyclo(DapOctanRRRRQ) | SEQ ID NO: 38 |
| CPP1-4 | cyclo(DapDecaRRRRQ) | SEQ ID NO: 39 |
| CPP1-5 | cyclo(Dap1-PyrenRRRRQ) | SEQ ID NO: 40 |
| CPP1-6 | cyclo(Dap3,3-diphenyRRRRQ) | SEQ ID NO: 41 |
| CPP1-7 | cyclo(DapFmocRRRRQ) | SEQ ID NO: 42 |
| CPP1-8 | cyclo(Dap1-PyrenebRRRRQ) | SEQ ID NO: 43 |
| CPP1-10 | cyclo(DapDecaRrRrQ) | SEQ ID NO: 44 |
| CPP1-11 | cyclo(DapDecarRrRQ) | SEQ ID NO: 45 |
| CPP1-12 | cyclo(DapDecaARRRQ) | SEQ ID NO: 46 |
| CPP1-13 | cyclo(DapDecaRRRAQ) | SEQ ID NO: 47 |
| CPP1-14 | cyclo(DapDecaRRRRRQ) | SEQ ID NO: 48 |
| CPP1-15 | cyclo(LysDecaRRRRQ) | SEQ ID NO: 49 |
| CPP1-16 | cyclo(DapDecaRRRQ) | SEQ ID NO: 50 |
| CPP1-17 | cyclo(OrnDecaRRRRQ) | SEQ ID NO: 51 |
| CPP1-18 | cyclo(LysDecaRrRrQ) | SEQ ID NO: 52 |
| CPP1-19 | cyclo(LysDecarRrRQ) | SEQ ID NO: 53 |
| CPP1-20 | cyclo(AspDecyRRRRQ) | SEQ ID NO: 54 |
| CPP1-22 | cyclo(AspDecyRrRrQ) | SEQ ID NO: 55 |
| CPP1-23 | cyclo(GluDecyRrRrQ) | SEQ ID NO: 56 |
| CPP1-24 | cyclo(AspDecyrRrRQ) | SEQ ID NO: 57 |
| CPP1-25 | cyclo(GluDecyrRrRQ) | SEQ ID NO: 58 |

*single letter abbreviations: when shown in capital letters herein it indicates the L-amino acid form, when shown in lower case herein it indicates the D-amino acid form. ϕ = 2-naphthylalanine, Dap = 2,3-diaminopropionic acid, tBu = tert-butanoyl, hexan = hexanoyl, octan = octanoyl, deca = decanoyl, 1-pyren = pyrenol, 3,3-diphenyl = 3,3-diphenoyl, Fmoc = fluorenylmethyloxycarbonoyl, 1-pyreneb = 1-pyrenylbutanoyl, decy = decynoyl

In some embodiments, the nanobody may be subjected to an alteration to render it less immunogenic when administered to a human. Such an alteration may comprise one or more of the techniques commonly known as chimerization, humanization, CDR-grafting, deimmunization and/or mutation of framework region amino acids to correspond to the closest human germline sequence (germlining). Bispecific antibodies which have been altered will therefore remain administrable for a longer period of time with reduced or no immune response-related side effects than corresponding bispecific antibodies which have not undergone any such alteration(s). One of ordinary skill in the art will understand how to determine whether, and to what degree a nanobody must be altered in order to prevent it from eliciting an unwanted host immune response.

Also disclosed herein is a polynucleotide molecule encoding the above nanobody or fragment or fusion protein thereof. Polynucleotides of the invention may be in the form of DNA or RNA. DNA forms include cDNA, genomic DNA, or synthetic DNA. DNA can be single-stranded or double-stranded. DNA can be a coding strand or a non-coding strand.

### Detectable Moiety

In some embodiments, the nanobody disclosed herein is conjugated to a detectable moiety. The detectable moiety can be any moiety that is capable of producing, either directly or indirectly, a detectable signal. Examples of detectable moieties for antibodies include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, beta-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance. Such antibodies and their fragments may be used for diagnostic applications, including but not limited to detection applications and imaging applications.

In some embodiments, a detectable moiety comprises a fluorophore. Representative fluorophores include, but are not limited to 7-dimethylaminocoumarin-3-carboxylic acid, dansyl chloride, nitrobenzodiazolamine (NBD), dabsyl chloride, cinnamic acid, fluorescein carboxylic acid, Nile Blue, tetramethylcarboxyrhodamine, tetraethyl sulfohodamine, 5-carboxy-X-rhodamine (5-ROX), and 6-carboxy-X-rhodamine (6-ROX). It is understood that these representative fluorophores are exemplary only, and additional fluorophores can also be employed. For example, there the ALEXA FLUOR® dye series includes at least 19 different dyes that are characterized by different emission spectra. These dyes include ALEXA FLUOR® 350, 405, 430, 488, 500, 514, 532, 546, 555, 568, 594, 610, 633, 635, 647, 660, 680, 700, and 750 (available from Invitrogen Corp., Carlsbad, Calif., United States of America), and the choice of which dye to employ can be made by the skilled artisan after consideration of the instant specification based on criteria including, but not limited to the chemical compositions of the specific ALEXA FLUOR©, whether multiple detectable moieties are to be employed and the emission spectra of each, the detection technique to be employed, etc.

In some embodiments, a detectable moiety is a cyanine dye. Non-limiting examples of cyanine dyes that can be conjugated to the antibody fragments of the presently disclosed subject matter include the succinimide esters CyS, CyS.5, and Cy7, supplied by Amersham Biosciences (Piscataway, N.J., United States of America).

In some embodiments, a detectable moiety comprises a near infrared (NIR) dye. Non-limiting examples of near infrared dyes that can be conjugated to the antibody fragment of the presently disclosed subject matter include NIR641, NIR664, NIT7000, and NIT782.

### Pharmaceutical composition

Also disclosed is a pharmaceutical composition comprising a disclosed nanobody in a pharmaceutically acceptable carrier. Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. PP. Gerbino, Lippincott Williams & Wilkins, Philadelphia, PA. 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutically acceptable carriers include any and all suitable solvents, dispersion media, coatings, antibacterial and antifungal agents, isotonicity agents, antioxidants and absorption delaying agents, and the like that are physiologically compatible with a bispecific antibody of the present invention. Examples of suitable aqueous and nonaqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, carboxymethyl cellulose colloidal solutions, tragacanth gum and injectable organic esters, such as ethyl oleate, and/or various buffers. Pharmaceutically acceptable carriers include sterile aqueous solutions or dispersions and sterile powders for the extemporaneous preparation of sterile injectable solutions or dispersion. Proper fluidity may be maintained, for example, by the use of coating materials, such as lecithin, by the maintenance of the required particle size in the case of dispersions, and by the use of surfactants.

Pharmaceutical nanobody may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole, butylated hydroxytoluene, lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmaceutical nanobody may also comprise isotonicity agents, such as sugars, polyalcohols, such as mannitol, sorbitol, glycerol or sodium chloride in the compositions.

The pharmaceutical nanobody may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The bispecific antibodies may be prepared with carriers that will protect the bispecific antibody against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well known in the art. Methods for the preparation of such formulations are generally known to those skilled in the art.

Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Also disclosed is the use of a disclosed nanobody for use as a medicament for the treatment of human monocytic ehrlichiosis (HME).

### Methods of Use

Also disclosed is a method for endoscopic visualization of target tissues, such as colorectal adenomas, in a subject by administering to the subject an effective amount of a disclosed nanobody conjugated to a detectable moiety (“nanobody composition”). In some embodiments, the method comprises administering to the subject a nanobody composition disclosed herein under conditions sufficient for binding the nanobody composition to a target tissue, and detecting the detectable moiety in the target tissue. In some embodiments of the method, a carboxyl group of the non-steroidal anti-inflammatory drug is derivatized to an ester or secondary amide.

In some embodiments, the target tissue is selected from the group consisting of an inflammatory lesion, a pre-neoplastic lesion, a tumor, a neoplastic cell, a pre-neoplastic cell, and a cancer cell. In some embodiments, the pre-neoplastic lesion is selected from the group consisting of a colon polyp. In some embodiments, the tumor is selected from the group consisting of a primary tumor, a metastasized tumor, and a carcinoma. In some embodiments, the target tissue is an aberrant crypt foci, hyperplastic polyp, macro-adenoma, micro-adenoma, or any combination thereof.

In some embodiments of the present method, the subject is a mammal. In some embodiments, the mammal is a human.

Various routes of administration of the imaging agent can be employed in the disclosed methods. In some embodiments, the administering is via a route selected from the group consisting of peroral, intravenous, intraperitoneal, inhalation, and intratumoral.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1. Discovery of Anti-COX-2 Nanobodies for Endoscopic Visualization of Colorectal Adenomas

### Abstract

Colorectal cancer (CRC) is one of the leading causes of cancer-related mortality in men and women. Uncontrolled proliferation of CRC cells is pivotal in colorectal tumorigenesis and cyclooxygenase-2 (COX-2) is an important regulatory enzyme in this process. Timely diagnosis is the key to life-saving management of CRC, which is under-diagnosed because colorectal aberrant crypt foci, hyper plastic polyps, micro edemas are often missed with conventional colonoscopy. Towards addressing this unmet medical need, an alpaca-derived library of “nanobodies” against COX-2 was discovered, which is overexpressed in early stages of colorectal carcinogenesis. Described here is one such nanobody, A7, that was conjugated to a NIR664 fluorophore at its N-terminal end without losing COX-2-selective binding with A7-NIR664. In vivo fluorescence endoscopic imaging with A7-NIR664 detects azoxymethane/dextran sodium sulfate-induced colorectal adenomas in mice after intravenous injection of A7-NIR664. This work demonstrates the 1st COX-2-targeted nanobodies and a fluorescent NIR derivative exhibiting high affinity for COX-2 enzyme and offering significant advantages over standard endoscopic imaging available in the clinic today.

### Introduction

Technologies for imaging colorectal cancer (CRC) in animal models and patients have been significantly improved through advances in instrumentation, enabling high-resolution visualization of tissue structure. Furthermore, decades of basic and clinical research have identified several molecular biomarkers that may be valuable for assessing susceptibility to CRC, and CRC progression, as well as dissecting molecular mechanisms of CRC pathophysiology in preclinical studies. Early diagnosis is the key to effective management of CRC that can save lives. Current CRC imaging strategies, including white light colonoscopy, chromoendoscopy, narrow-band imaging, aim to detect and enable surgical resection of solid tumors and precursor lesions (Abegunde, AT. Clin Gastroenterol Hepatol 2016 14:1062). Using these methods, raised solid tumors and mature CRC can typically be visualized and removed, whereas aberrant crypt foci, hyperplastic polys, and macro- or micro-adenomas are frequently missed (Zhao, S, et al. Gastroenterology 2019 156(6):1661-1674.e11; van Rijn, JC, et al. Am J Gastroenterol 2006 101:343-350). This is clearly an unmet medical need that warrants critical development of novel strategies enabling early diagnostic imaging of CRC in preclinical and clinical settings. To date, most efforts to develop early CRC imaging agents have been peptides or antibodies targeting surface receptors e.g., the epidermal growth factor receptor or enzymes in the extracellular milieu e.g., matrix metalloproteinases (Goetz, M., et al. Gastroenterology 2010 138:435-446; Liu, J, et al. Cancer Lett 2013 330:200-207; Yoon, S M, et al. Gut Liver 2010 4:488-497). Although shown to have promise, these reagents remain at the pre-clinical stages of development.

The cyclooxygenase-2 (COX-2) overexpression is one of the major driving forces of colorectal tumorigenesis that makes COX-2 protein a promising imaging target for the development of diagnostic agents for early CRC. It was hypothesize that imaging agents tagged to nanobodies specific for the CRC biomarker COX-2 would selectively accumulate in colorectal adenomas, resulting in specific and sensitive early detection, while reducing background and off-target systemic toxicity. In prior studies, in vivo imaging of COX-2 was shown in multiple animal models of premalignant and malignant tumors and inflammation using small molecule fluorescently-or radio-labeled targeted agents (Uddin, M J, et al. Cancer Res 2010 70:3618-3627; Uddin, M J, et al. Bioconjug Chem 2013 24:712-723; Uddin, M J, et al. ACS Med Chem Lett 2014 5:446-450; Uddin, M J, et al. Biomaterials 2016 92:1-80; Uddin, M J, et al. ACS Med Chem Lett 2020 11:1875-1880; Uddin, M J, et al. J Biomed Opt 2016 21:90503). These studies provide proof-of-principle for targeted molecular imaging of COX-2 in pathogenesis in vivo.

Monoclonal antibodies (mAbs) are large-sized molecules (˜150 kDa) and exhibit limited ability for tumor penetration. However, nanobodies (˜15 kDa) are the smallest functional antigen binding fragments derived from the naturally occurring heavy-chain-only antibodies capable of cell and tissue penetration that were first identified in the blood of llamas (Hamers-Casterman, C, et al. Nature 1993 363:446-448). Targeted nanobodies are particularly interesting because they are water soluble for intravenous injection, unlike small molecule inhibitors with solubility issues so they warrant nano-formulation for systemic dosing. This Example reports the discovery of a COX-2-targeted nanobody and nanobody-based fluorescence imaging agents capable of targeting COX-2 in mouse colorectal adenomas. The immunoprobe combines structural and functional imaging readouts obtained with fluorescence-aided colonoscopy measurements providing powerful diagnostic information for early detection and improved management of colorectal carcinogenesis.

### Results

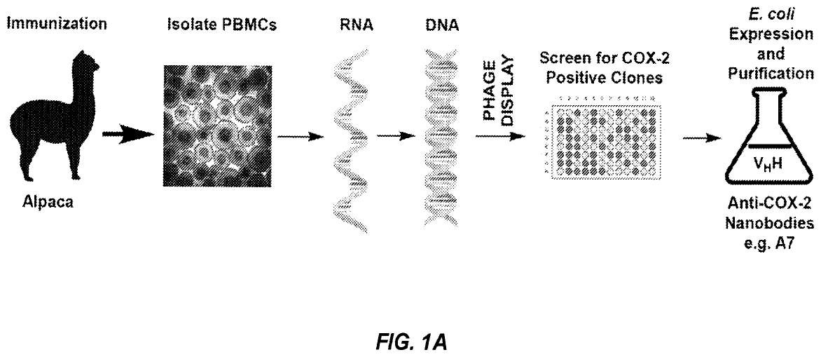

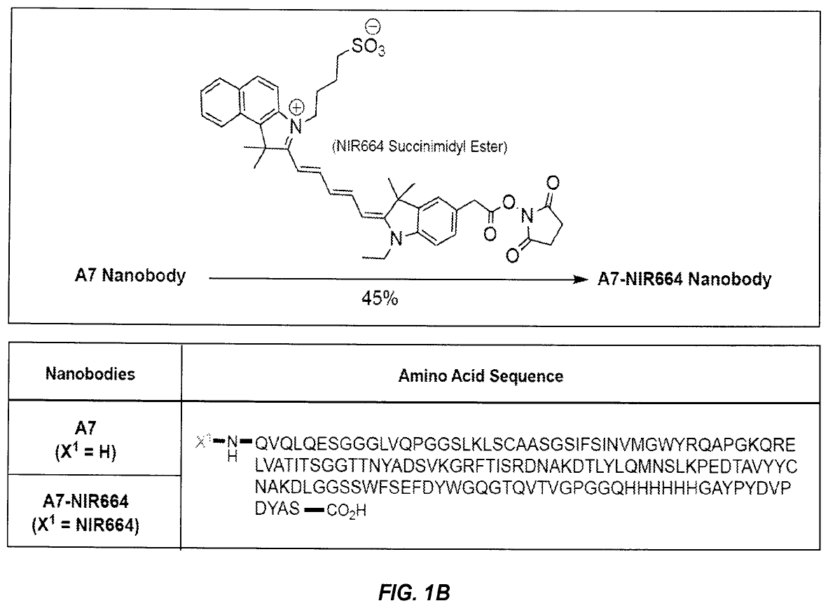

In this protocol, peripheral blood mononuclear cells (PBMCs) were purified from alpacas immunized with murine COX-2 (mCOX-2) enzyme. Using mRNA isolated from these PBMCs, the entire library of single chain variable regions was amplified by reverse transcription polymerase chain reaction (RT-PCR) and cloned into a phagemid vector that results in fusion of the antibody gene to a coat protein of m13 bacteria phage. A promising clone, called “A7”, was isolated from VHH sequences produced in the phage library against the antigen, murine COX-2. The native A7 and F9 nanobody clones were expressed in E. coli cells. The purification of expressed A7 and F9 nanobodies was performed using a size-exclusion (SEC) column on a fast protein liquid chromatography (FPLC) system. Then, NIR664 (excitation λₘₐₓ 670 nm and emission λₘₐₓ 690 nm) fluorophore was conjugated to the N-terminus end of A7 nanobody in phosphate buffered saline. The fluorescently conjugated A7-NIR664 nanobody was purified using an FPLC system (FIG. 1A-1B).

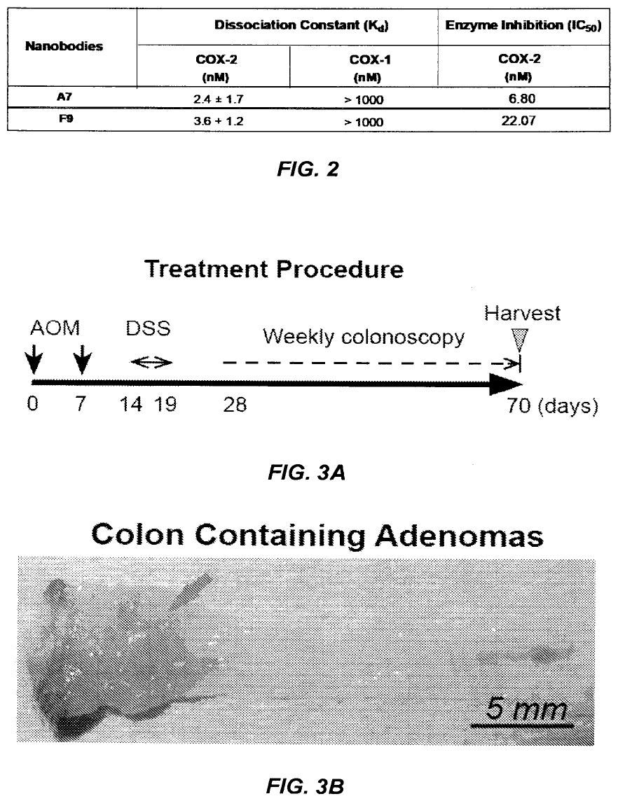

The dissociation constant of A7 and F9 nanobodies while bound to apo mCOX-2 was evaluated by a microscale thermophoresis (MST) assay (Bartoschik, T, et al. Sci Rep 2018 8:4977). In this assay, A7 and F9 nanobodies were identified as high affinity COX-2 binding nanobodies with dissociation constants (Kd) of 2.4±1.7 nM and 3.6±1.2 nM, respectively. In a similar assay, where hCOX-1 enzyme was used in place of COX-2, both A7 and F9 exhibited dissociation constants of >1 μM, suggesting that it have no affinity for COX-1 (FIG. 2). Further, the COX-2 inhibitory activity of A7 and F9 nanobodies was evaluated in the production of prostaglandin E2 (PGE2) and prostaglandin D2 (PGD2) using arachidonic acid (AA) as a substrate. In this assay, purified mouse COX-2 expressed in baculovirus-infected Sf9 cells was used. Purified COX-2 was incubated with varying concentrations of test nanobodies followed by the addition of hematin and AA, respectively. The PGE2 and PGD2 products in the reaction was quantified by a tandem liquid chromatography mass spectroscopy (LC-MS/MS) analysis. In this assay, A7 and F9 nanobodies exhibited IC50 values of 6.8 nM and 22.07 nM, respectively (FIG. 2). The results suggest that binding of nanobody induces a conformational change in the COX-2 protein to inhibit the production of PGE2 and PGD2.

Murine macrophage-like RAW264.7 cells were plated on 35 mm cell culture dishes (MatTek Corporation, Ashland, MA) such that the cells were 50% confluent on the day of the experiment. The RAW264.7 cells were activated for 8 hours in serum-free DMEM with 200 ng/ml of di[3-deoxy-D-manno-octulosonyl]-lipid A (KLA). Cells were incubated in 2.0 ml HBSS with A7-NIR664 [200 nM] conjugate [λₑₓ=670 nm and λₑₘ=690 nm) for 1 hour at 37° C. The treated cells were then briefly washed three times with HBSS. Following washout period, the cells were imaged in 2.0 ml fresh HBSS on a Zeiss Axiovert 25 Microscope with the propidium iodide filter (0.5-1.0 sec exposure, gain of 2). The A7-NIR664 nanobody excites at 670 nm and emits at 690 nm wavelengths. All treatments were performed in duplicate dishes in at least three separate experiments. FIG. 5 displays the fluorescence image of KLA-activated or non-activated RAW264.7 cells incubated with A7-NIR664. The imaging shows that the KLA-activated RAW264.7 cells exhibited strong labeling with A7-NIR664 nanobody (FIG. 5A). Importantly, no labeling was observed when non-activated RAW264.7 cells that do not express COX-2, were incubated with A7-NIR664 (FIG. 5B). Results from this experiment suggest that A7-NIR664 nanobody is selective for COX-2-expressing cells, and the uptake and retention of the fluorescent nanobody in the KLA-activated RAW264.7 cells are COX-2-dependent.

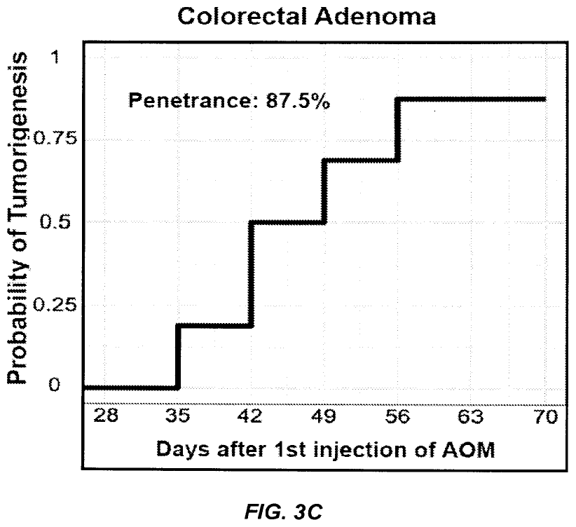

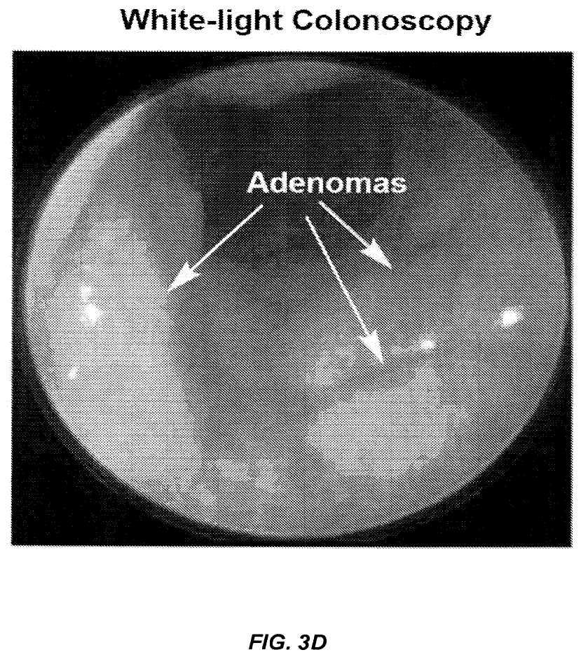

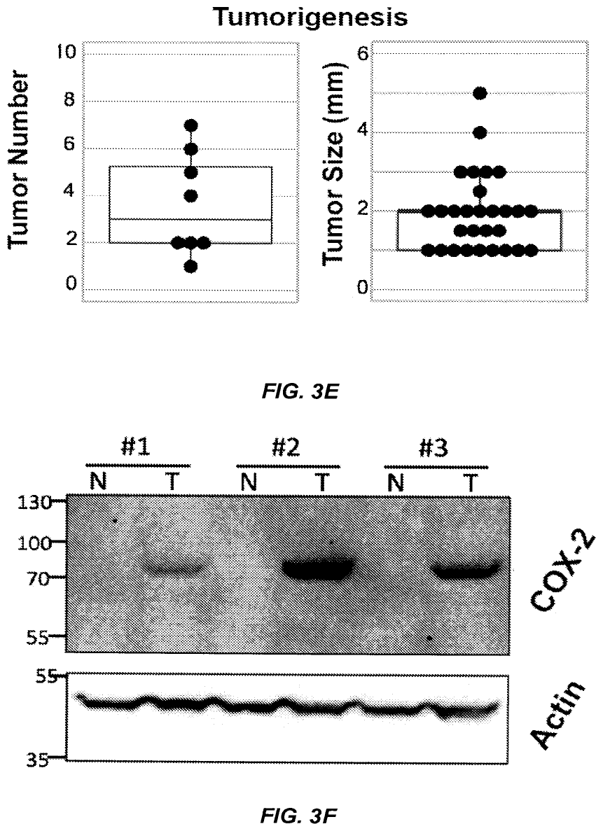

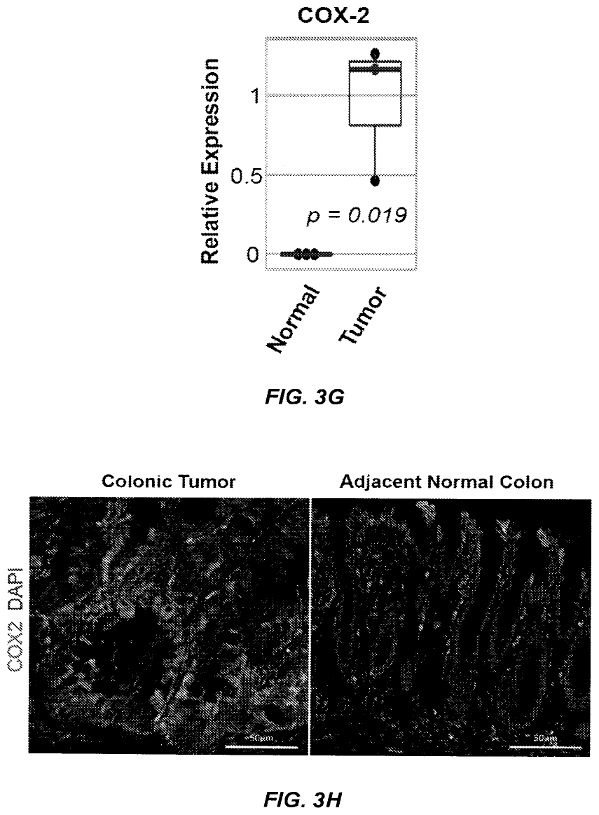

Azoxymethane (AOM)/dextran sodium sulfate (DSS)-induced colorectal adenomas was developed in B6;129 mice. In this model, colorectal adenomas in the treated animals are typically observed at 8 weeks post-AOM administration. The AOM/DSS model recapitulates a multistep progression from colonic dysplasia to micro- and macro-adenomas, and ultimately carcinomas that mimics human colorectal cancer, although the genetic aberrations associated with the neoplasia are much more diverse than those found in human patients. AOM/DSS-induced tumors are located predominantly in the distal colon, which can be easily accessed by a colonoscope, and are macroscopically flat, nodular, or polypoid, coinciding with the morphological diversity of human colorectal tumors (Pan Q, et al. Sci Rep 2017 7:25; Suzuki, R, et al. Cancer Sci 2004 95:721-727) (FIG. 3A-3E). An immunoblotting assay (FIG. 3F-3G) and an immunofluorescence assay (FIG. 3H) were used to evaluate COX-2 expression in the adenoma tissues. The results showed high COX-2 expression in AOM/DSS-induced mouse colorectal adenomas as compared to the adjacent normal colon. This AOM/DSS colon adenoma model provides an excellent opportunity to evaluate the ability of A7-NIR664 aiding early detection of CRC in vivo.

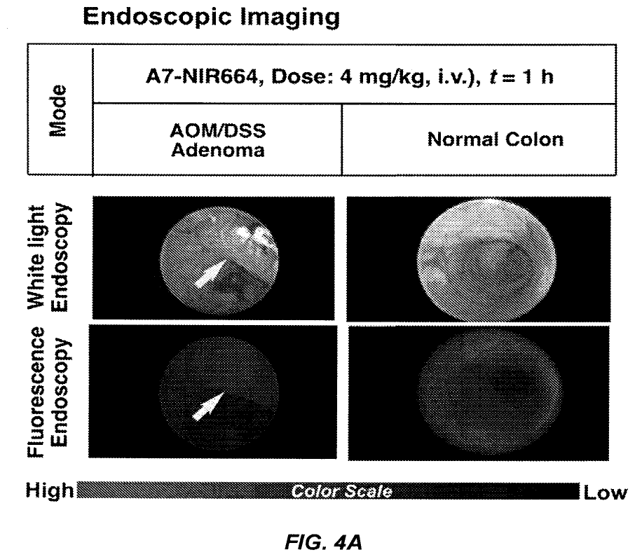

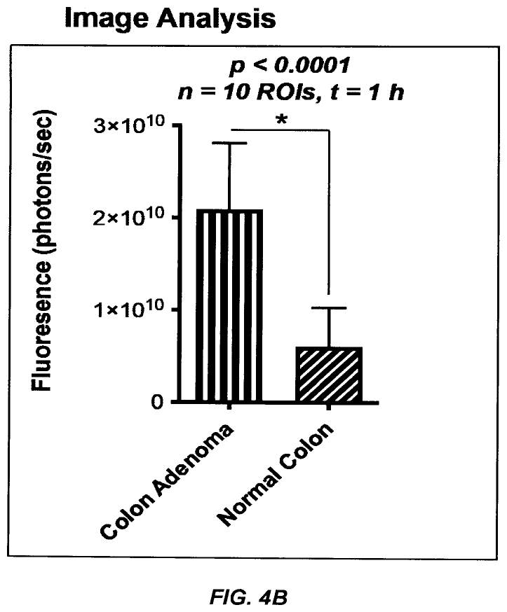

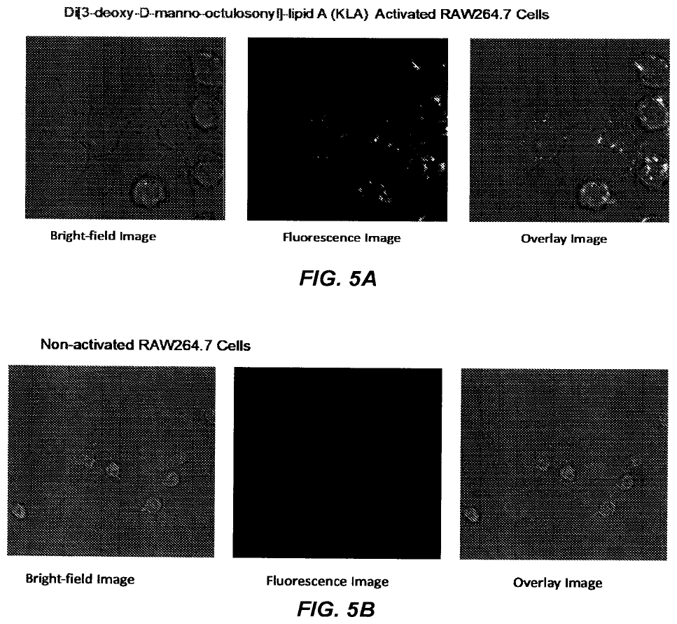

Finally, the ability of A7-NIR664 nanobody in targeting COX-2 and lighting up AOM/DSS-induced colorectal adenomas was evaluated in B6; 129 mice. In this experiment, B6;129 mice bearing colorectal adenomas were injected intravenously with A7-NIR664 (4 mg/kg dose) prior to fluorescence colonoscopy enabled a clear delineation of colorectal adenomas at 30 min to 2 h post-administration. The imaging data showed a homogeneous distribution of the probe throughout the tumors with significantly high tumor uptake compared to that of the surrounding normal tissues (FIG. 4A). The signal-to-noise ratios were determined by image analysis using ImageJ software as ˜7 (p<0.001, n=4 ROIs, t=1 h) (FIG. 4B).

### Discussion

Most efforts to develop imaging agents for visualization of CRC have been peptides or antibodies targeting surface receptors (e.g., the epidermal growth factor receptor) or enzymes in the extracellular milieu (e.g., matrix metalloproteinases) (Goetz, M., et al. Gastroenterology 2010 138:435-446; Liu, J, et al. Cancer Lett 2013 330:200-207; Yoon, SM, et al. Gut Liver 2010 4:488-497). Although shown to have promise, these reagents remain at the pre-clinical stages of development. This might be due to the disadvantages associated with these compounds, such as potential metabolic instability, poor tissue penetration, poor pharmacokinetics, and immunogenicity. Nanobodies that target an intracellular enzyme, such as COX-2, thereby avoiding these potential pitfalls. Although the use of COX-2 as a target for imaging in cancer has been pursued previously, in most cases, positron-emitting nuclides were incorporated into the structures of known COX-2 inhibitors for use as PET tracers (de Vries, E F, J Nucl Med 2003 44:1700-1706; Huang, Y C, et al. Molecules 2016 21:387; Tietz, O, et al. Nucl Med Biol 2018 62-63:9-17; Tietz, O, et al. EJNMMI Res 2016 6:37; Uddin, M J et al. Cancer Prev. Res. 2011 4:1536-1545; Wuest, F. Bioorg Med Chem 2008 16:7662-7670). Such agents are not compatible with colonoscopy and not suited for application to reduce the incidence of adenomas with the current standard screening approach. Fluorescently labeled small molecules have been reported in the literature for COX-2-targeted optical imaging (Tondera, C, et al. Biochem Biophys Res Commun 2015 458:40-45; Zhang, H, et al. J Am Chem Soc 2013 135:11663-11669; A. Bhardwaj, ChemMedChem 2014 9:109-116); however, the applicability of this molecule to imaging in vivo is limited by their optical properties.

The disclosed nanobody-based approach for early CRC constitutes an entirely new and innovative way to precisely target COX-2 via endoscopic fluorescence imaging. We developed a novel series of alpaca-derived nanobodies against the COX-2 enzyme. From a panel of 73 nanobodies derived from a combined naïve Camelid phage display library against native COX-2, A7 was discovered as a high affinity COX-2 binding nanobody. Endoscopic visualization of colorectal adenomas in mice using A7-NIR664 showed clear delineation of adenomas of the colon. The fluorescent COX-2 nanobody represents the smallest functional antigen binding fragments derived from the naturally occurring heavy-chain-only antibodies. This A7 nanobody is particularly important, because: a) it allows easy expression, b) it binds tightly with its target COX-2, c) it tolerates conjugation of a bulky fluorophore retaining target binding properties, d) it is stable in circulation and COX-2-specific, e) it penetrates through the cell membrane efficiently, f) it binds tightly to the targeted adenoma cells in intact animals, g) it distributes through the bloodstream to reach the regions of interest, and h) it targets COX-2 in colorectal adenomas and allows acquisition of images with high tumor-to-background contrast after systemic administration.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising a nanobody, wherein the nanobody comprises CDR1, CDR2, and CDR3 sequences, wherein the CDR1 sequence comprises the amino acid sequence GSIFSINVM (SEQ ID NO:1), CDR2 sequence of the variable domain comprises the amino acid sequence ELVATITSGGTTNY (SEQ ID NO:2), and the CDR3 sequence of the variable domain comprises the amino acid sequence VYYCNAKDLGGSSWFSEFDY (SEQ ID NO: 3).

2. The composition of claim 1, wherein the nanobody comprises the amino acid sequence SEQ ID NO:4.

3. The composition of claim 1, wherein the nanobody is conjugated to a detectable moiety.

4. The composition of claim 3, wherein the moiety is a near infrared (NIR) dye.

5. The composition of claim 4, wherein the NIR is NIR641, NIR664, NIT7000, NIT782, IRDye800, or ICG.

6. A method for endoscopic visualization of a colorectal adenoma in a subject, comprising administering to the subject an effective amount of the composition of claim 3, and endoscopically detecting the moiety in the subject.

7. The method of claim 6, wherein the colorectal adenoma is an aberrant crypt foci, hyperplastic polyp, macro-adenoma, micro-adenoma, or any combination thereof.


SEQUENCE LISTING (59 sequences)

SEQ ID NO: 1 - protein, 9 aa, synthetic construct
GSIFSINVM

SEQ ID NO: 2 - protein, 14 aa, synthetic construct
ELVATITSGGTTNY

SEQ ID NO: 3 - protein, 20 aa, synthetic construct
VYYCNAKDLGGSSWFSEFDY

SEQ ID NO: 4 - protein, 125 aa, synthetic construct
QVQLQESGGGLVQPGGSLKLSCAASGSIFSINVMGWYRQAPGKQRELVATITSGGTTNYADSVKGRFTISRDNAKDTLYLQMNSLKPEDTAVYYCNAKDLGGSSWFSEFDYWGQGTQVTVGPGGQ

SEQ ID NO: 5 - protein, 143 aa, synthetic construct
QVQLQESGGGLVQPGGSLKLSCAASGSIFSINVMGWYRQAPGKQRELVATITSGGTTNYADSVKGRFTISRDNAKDTLYLQMNSLKPEDTAVYYCNAKDLGGSSWFSEFDYWGQGTQVTVGPGGQHHHHHHGAYPYDVPDYAS

SEQ ID NO: 6 - DNA, 436 bp, synthetic construct
  source 1..436: mol_type = other DNA
atggcccaggtgcagctgcaggagtctggaggaggcttggtgcagcctggggggtctctgaagctctcctgtgcagcctctggaagcatcttcagtatcaatgtcatgggctggtaccgccaggctccagggaagcagcgcgagttggtcgcaactattactagtggtggtaccacaaactatgcagactccgtgaagggccgattcaccatctccagagacaacgccaaggacacgctgtatctgcaaatgaacagcctgaaacctgaggacacggccgtctattactgtaatgccaaagacttgggcggtagtagctggtttagtgagtttgactactggggccagggaacccaggtcaccgtcggcccgggaggccaacaccatcaccaccatcatggcgcatatccgtatgatgtgccggactatgcttctt

SEQ ID NO: 7 - protein, 8 aa, synthetic construct
IRRGISRK

SEQ ID NO: 8 - protein, 6 aa, synthetic construct
KRKRAV

SEQ ID NO: 9 - protein, 9 aa, synthetic construct
PKPKRQTKR

SEQ ID NO: 10 - protein, 8 aa, synthetic construct
RRRRHCNR

SEQ ID NO: 11 - protein, 8 aa, synthetic construct
IPDPTGQS

SEQ ID NO: 12 - protein, 8 aa, synthetic construct
IKREREND

SEQ ID NO: 13 - protein, 17 aa, synthetic construct
KKLQEQEKQQKVEFRKR

SEQ ID NO: 14 - protein, 9 aa, synthetic construct
GPNKKKRKL

SEQ ID NO: 15 - protein, 19 aa, synthetic construct
RRRRASAPISQWSSSRRSR

SEQ ID NO: 16 - protein, 18 aa, synthetic construct
KLALKLALKALKAALKLA

SEQ ID NO: 17 - protein, 13 aa, synthetic construct
FLPLIGRVLSGIL

SEQ ID NO: 18 - protein, 26 aa, synthetic construct
CVQWSLLRGYQPCCVQWSLLRGYQPC

SEQ ID NO: 19 - protein, 32 aa, synthetic construct
RKKRKGSGSRKKRKGSGSRKKRKGSGSRKKRK

SEQ ID NO: 20 - protein, 30 aa, synthetic construct
CVQWSLLRGYQPCGSGSCVQWSLLRGYQPC

SEQ ID NO: 21 - protein, 32 aa, synthetic construct
KKRRRGSGSKKRRRGSGSKKRRRGSGSKKRRR

SEQ ID NO: 22 - protein, 32 aa, synthetic construct
RKKRRGSGSRKKRRGSGSRKKRRGSGSRKKRR

SEQ ID NO: 23 - protein, 17 aa, synthetic construct
RQIKIWFQNRRMKWKKC

SEQ ID NO: 24 - protein, 28 aa, synthetic construct
GRKRKRSGRKRKRSGRKRKRSGRKRKRS

SEQ ID NO: 25 - protein, 28 aa, synthetic construct
PRKKRGRPRKKRGRPRKKRGRPRKKRGR

SEQ ID NO: 26 - protein, 13 aa, synthetic construct
CVQWSLLRGYQPC

SEQ ID NO: 27 - protein, 9 aa, synthetic construct
  VARIANT 1: note = Xaa is Arg or Lys
  VARIANT 3: note = Xaa is Arg or Lys
  VARIANT 5: note = Xaa is Arg or Lys
XKXRXGSGS

SEQ ID NO: 28 - protein, 6 aa, synthetic construct
HHHHHH

SEQ ID NO: 29 - protein, 9 aa, synthetic construct
RKKRRQRRR

SEQ ID NO: 30 - protein, 12 aa, synthetic construct
RQARRNRRRRWR

SEQ ID NO: 31 - protein, 7 aa, synthetic construct
PKKKRKV

SEQ ID NO: 32 - protein, 12 aa, synthetic construct
HHHHHHHHHHHH

SEQ ID NO: 33 - protein, 7 aa, synthetic construct
  SITE 2: note = 2-naphthylalanine
  REGION 1: note = peptide is circular
FXRRRRQ

SEQ ID NO: 34 - protein, 7 aa, synthetic construct
  REGION 1: note = peptide is circular
  SITE 2: note = 2-naphthylalanine
  SITE 1: note = D-phenylalanine
  SITE 4: note = D-arginine
  SITE 6: note = D-arginine
FXRRRRQ

SEQ ID NO: 35 - protein, 8 aa, synthetic construct
  REGION 1: note = peptide is circular
  SITE 3: note = 2-naphthylalanine
  SITE 2: note = D-phenylalanine
  SITE 5: note = D-arginine
  SITE 7: note = D-arginine
FFXRRRRQ

SEQ ID NO: 36 - protein, 7 aa, synthetic construct
  REGION 1: note = peptide is circular
  SITE 2: note = tert-butanoyl
FXRRRRQ

SEQ ID NO: 37 - protein, 7 aa, synthetic construct
  REGION 1: note = peptide is circular
  SITE 1: note = 2,3-diaminopropionic acid
  SITE 2: note = hexanoyl
XXRRRRQ

SEQ ID NO: 38 - protein, 7 aa, synthetic construct
  REGION 1: note = peptide is circular
  SITE 1: note = 2,3-diaminopropionic acid
  SITE 2: note = octanoyl
XXRRRRQ

SEQ ID NO: 39 - protein, 7 aa, synthetic construct
  REGION 1: note = peptide is circular
  SITE 1: note = 2,3-diaminopropionic acid
  SITE 2: note = decanoyl
XXRRRRQ

SEQ ID NO: 40 - protein, 7 aa, synthetic construct
  REGION 1: note = peptide is circular
  SITE 1: note = 2,3-diaminopropionic acid
  SITE 2: note = pyrenol
XXRRRRQ

SEQ ID NO: 41 - protein, 7 aa, synthetic construct
  REGION 1: note = peptide is circular
  SITE 1: note = 2,3-diaminopropionic acid
  SITE 2: note = 3,3-diphenoyl
XXRRRRQ

SEQ ID NO: 42 - protein, 7 aa, synthetic construct
  REGION 1: note = peptide is circular
  SITE 1: note = 2,3-diaminopropionic acid
  SITE 2: note = fluorenylmethyloxycarbonoyl
XXRRRRQ

SEQ ID NO: 43 - protein, 7 aa, synthetic construct
  REGION 1: note = peptideis circular
  SITE 1: note = 2,3-diaminopropionic acid
  SITE 2: note = 1-pyrenylbutanoyl
XXRRRRQ

SEQ ID NO: 44 - protein, 7 aa, synthetic construct
  REGION 1: note = peptide is circular
  SITE 1: note = 2,3-diaminopropionic acid
  SITE 2: note = decanoyl
  SITE 4: note = D-arginine
  SITE 6: note = D-arginine
XXRRRRQ

SEQ ID NO: 45 - protein, 7 aa, synthetic construct
  REGION 1: note = peptide is circular
  SITE 1: note = 2,3-diaminopropionic acid
  SITE 2: note = decanoyl
  SITE 3: note = D-argine
  SITE 5: note = D-arginine
XXRRRRQ

SEQ ID NO: 46 - protein, 7 aa, synthetic construct
  REGION 1: note = peptide is circular
  SITE 1: note = 2,3-diaminopropionic acid
  SITE 2: note = decanoyl
XXARRRQ

SEQ ID NO: 47 - protein, 7 aa, synthetic construct
  REGION 1: note = peptide is circular
  SITE 1: note = 2,3-diaminopropionic acid
  SITE 2: note = decanoyl
XXRRRAQ

SEQ ID NO: 48 - protein, 8 aa, synthetic construct
  REGION 1: note = peptide is circular
  SITE 1: note = 2,3-diaminopropionic acid
  SITE 2: note = decanoyl
XXRRRRRQ

SEQ ID NO: 49 - protein, 7 aa, synthetic construct
  REGION 1: note = peptide is circular
  SITE 2: note = decanoyl
KXRRRRQ

SEQ ID NO: 50 - protein, 6 aa, synthetic construct
  REGION 1: note = peptide is circular
  SITE 1: note = 2,3-diaminopropionic acid
  SITE 2: note = decanoyl
XXRRRQ

SEQ ID NO: 51 - protein, 7 aa, synthetic construct
  REGION 1: note = peptide is circular
  SITE 2: note = decanoyl
OXRRRRQ

SEQ ID NO: 52 - protein, 7 aa, synthetic construct
  REGION 1: note = peptide is circular
  SITE 2: note = decanoyl
  SITE 4: note = D-arginine
  SITE 6: note = D-arginine
KXRRRRQ

SEQ ID NO: 53 - protein, 7 aa, synthetic construct
  REGION 1: note = peptide is circular
  SITE 2: note = decanoyl
  SITE 3: note = D-arginine
  SITE 5: note = D-arginine
KXRRRRQ

SEQ ID NO: 54 - protein, 7 aa, synthetic construct
  REGION 1: note = peptide is circular
  SITE 2: note = decynoyl
DXRRRRQ

SEQ ID NO: 55 - protein, 7 aa, synthetic construct
  REGION 1: note = peptide is circular
  SITE 2: note = decynoyl
  SITE 4: note = D-arginine
  SITE 6: note = D-arginine
DXRRRRQ

SEQ ID NO: 56 - protein, 7 aa, synthetic construct
  REGION 1: note = peptide is circular
  SITE 2: note = decynoyl
  SITE 4: note = D-arginine
  SITE 6: note = D-arginine
EXRRRRQ

SEQ ID NO: 57 - protein, 7 aa, synthetic construct
  REGION 1: note = peptide is circular
  SITE 2: note = decynoyl
  SITE 3: note = D-arginine
  SITE 5: note = D-arginine
DXRRRRQ

SEQ ID NO: 58 - protein, 7 aa, synthetic construct
  REGION 1: note = peptide is circular
  SITE 2: note = decynoyl
  SITE 3: note = D-arginine
  SITE 5: note = D-arginine
EXRRRRQ

SEQ ID NO: 59 - protein, 143 aa, synthetic construct; identical to SEQ ID NO: 5